(19) United States
(12) Patent Application Publication
(10) Pub. No.: US 2025/0042990 A1
(43) Pub. Date: Feb. 6, 2025

(54) ANTI-IL-1-BETA ANTIBODIES

(71) Applicant: Eli Lilly and Company, Indianapolis, IN (US)

(72) Inventors: Supratik DUTTA, Indianapolis, IN (US); Juqun SHEN, Indianapolis, IN (US); Andrew Dixon SKORA, Indianapolis, IN (US); Cheng WANG, Indianapolis, IN (US)

(21) Appl. No.: 18/692,113

(22) PCT Filed: Sep. 12, 2022
(86) PCT No.: PCT/US2022/076267

§ 371 Date: Mar. 14, 2024

Related U.S. Application Data
(60) Provisional application No. 63/246,036, filed on Sep. 20, 2021.

(51) Int. Cl. C07K 16/24; A61K 39/00; A61P 29/00

(52) U.S. Cl. CPC C07K 16/245; A61P 29/00; A61K 2039/505; C07K 2317/14; C07K 2317/33; C07K 2317/565; C07K 2317/76; C07K 2317/92

(57) ABSTRACT

The present invention provides engineered human interleukin-1 beta antibodies, cells and vectors comprising DNA encoding the same, and methods for producing the antibodies. In addition, the present invention provides the use of the human engineered interleukin-1-beta antibodies for the treatment of inflammatory disease, such as cardiovascular disease and cancer.

The present invention relates to the field of medicine. More particularly, the present invention relates to antibodies that bind human IL-1B (IL-1-beta or IL-1β or Interleukin-1ß have the same meaning herein) and may be useful for the treatment and/or prevention of inflammatory diseases, including but not limited to atherosclerotic cardiovascular disease (ASCVD), heart failure, cancer, and rare inherited disorders due to overproduction of IL-1B. The present invention is also related to methods of treating and/or preventing these inflammatory diseases.

Cardiovascular disease (CVD) is a class of diseases that involve the heart or blood vessels. Common manifestations of CVD include angina, myocardial infarction (MI, commonly known as a heart attack), stroke, heart failure, and arrythmia, among others. Because of the complex nature of the disease, many risk factors have been identified that contribute to initiation and progression of the disease. These include dyslipidemia, hypertension, diabetes, tobacco use, unhealthy diet, physical inactivity, and obesity. However, despite the efforts in controlling these traditional risk factors, cardiovascular disease remains the leading cause of death worldwide.

Research in the last two decades has emphasized the inflammatory process as a key component in the pathogenesis of CVD, particularly ASCVD. Epidemiologic data from the mid-1990s indicated that inflammation, as measured either by high-sensitivity C-reactive protein (hsCRP) or interleukin-6 (IL-6), was strongly associated with future major adverse cardiovascular events (MACE) in both primary and secondary prevention, independent of the traditional risk factors (Ridker et al. (2018) J. Am. Coll. Cardiol. 72: 3320-3331). Preclinical research has also demonstrated the role of inflammation in atherosclerotic plaque initiation and progression (Aday et al. (2019) Front. Cardiovasc. Med. 6:16 doi: 10.3389 fcvm.2019.00016). Importantly, inflammation also contributes to plaque destabilization and rupture, precipitating acute cardiovascular events such as MI and stroke.

The Interleukin-1 family is a pivotal element of inflammation and has been well studied as a therapeutic target for various inflammatory diseases (Szekely et al. (2018) Cardiol. Ther. 7:25-44). There are three members of the IL-1 gene family: IL-1α, IL-1β, and IL-1 receptor antagonist (IL-1ra). IL-1α, and IL-1β are agonists of the IL-1 receptor whereas the IL-1ra is a specific receptor antagonist and thus, an endogenous competitive inhibitor of IL-1. IL-1β is the primary circulating form of IL-1. It is produced as a precursor (pro-IL-1β) that is activated via the NLRP3 (NOD-, LRR- and pyrin domain-containing protein 3) inflammasome under a variety of inflammatory stimuli. The active form of IL-1β has autocrine, paracrine, and endocrine effects and, thus, is involved in a broad spectrum of inflammatory disorders.

IL-1β inhibition may also have a role in treatment of cancers that have an inflammatory basis. Many malignancies arise in areas of chronic inflammation, and inadequate resolution of inflammation could have a major role in tumor invasion, progression, and metastases (Grivennikov et al. (2010) Cell 140:883-899). Inflammation is of pathophysiological relevance in lung cancer; for example, smoking and other external inhaled toxins trigger persistent inflammatory response. This inflammatory activation is partly mediated through activation of the NLRP3 inflammasome, with local generation of active IL-1β. In the clinic, high baseline concentrations of hsCRP and IL-6 have been found to be associated with subsequently diagnosed lung cancer. IL-1β blockade with canakinumab was associated with reduction in total cancer mortality, incident lung cancer and lung cancer mortality (Ridker et al. (2017) Lancet 390:1833-1842).

There remains a need to provide therapeutic antibodies that bind human IL-1β. In particular, there remains a need to provide IL-1β antibodies that have favorable clinical attributes. The present invention encompasses engineered human antibodies against human IL-1β. The antibodies of the present invention display potent IL-1β neutralizing activity and are highly specific for IL-1β.

Accordingly, in some embodiments the present invention provides antibodies that bind human Protein IL-1β (SEQ ID NO:27), which comprise a heavy chain variable region (VH) and a light chain variable region (VL), wherein the VH comprises heavy chain complementarity determining regions (HCDR) HCDR1, HCDR2, and HCDR3, and the VL comprises light chain complementarity determining regions (LCDR) LCDR1, LCDR2, and LCDR3, wherein
the HCDR1 comprises AASGFTFSSYSX₁S, wherein X₁ is Phe or Leu (SEQ ID NO:8),
the HCDR2 comprises AISVSGGSTYYAX₂SVKG, wherein X₂ is Pro or Asp (SEQ ID NO:9),
the HCDR3 comprises ARDDLIX₃TRGTFYNWFDP, wherein X₃ is Thr or Pro (SEQ ID NO:10),
the LCDR1 comprises RASQSISSNLN (SEQ ID NO:12),
the LCDR2 comprises YAASSLQS (SEQ ID NO:13), and
the LCDR3 comprises QQSYSIPWT (SEQ ID NO:14).

Accordingly, in some embodiments the present invention provides antibodies that bind human Protein IL-1β, wherein
the HCDR1 comprises AASGFTFSSYSX₁S (SEQ ID NO:8), wherein X₁ is Phe,
the HCDR2 comprises AISVSGGSTYYAX₂SVKG (SEQ ID NO:9), wherein X₂ is Pro,
the HCDR3 comprises ARDDLIX₃TRGTFYNWFDP (SEQ ID NO:10), wherein X₃ is Thr.

Accordingly, in some embodiments the present invention provides antibodies that bind human Protein IL-1β, wherein
the HCDR1 comprises AASGFTFSSYSX₁S (SEQ ID NO:8), wherein X₁ is Leu,
the HCDR2 comprises AISVSGGSTYYAX₂SVKG (SEQ ID NO:9), wherein X₂ is Asp,
the HCDR3 comprises ARDDLIX₃TRGTFYNWFDP (SEQ ID NO:10), wherein X₃ is Pro.

Accordingly, in some embodiments the present invention provides antibodies that bind human Protein IL-1β, wherein
the HCDR1 comprises AASGFTFSSYSX₁S (SEQ ID NO:8), wherein X₁ is Leu,
the HCDR2 comprises AISVSGGSTYYAX₂SVKG (SEQ ID NO:9), wherein X₂ is Asp,
the HCDR3 comprises ARDDLIX₃ TRGTFYNWFDP (SEQ ID NO:10), wherein X₃ is Thr.

Accordingly, in some embodiments the present invention provides antibodies that bind human Protein IL-1β, wherein
the HCDR1 comprises AASGFTFSSYSX₁S (SEQ ID NO:8), wherein X₁ is Phe,
the HCDR2 comprises AISVSGGSTYYAX₂SVKG (SEQ ID NO:9), wherein X₂ is Asp,
the HCDR3 comprises ARDDLIX₃TRGTFYNWFDP (SEQ ID NO:10), wherein X₃ is Thr.

In some embodiments, the present invention provides an antibody, wherein the VH comprises a sequence selected from SEQ ID NO:7, SEQ ID NO:17, SEQ ID NO:22, or SEQ ID NO:25 and the VL comprises SEQ ID NO:11. In other embodiments, the present invention provides an antibody, wherein the VH consists of a sequence selected from SEQ ID NO:7, SEQ ID NO:17, SEQ ID NO:22, or SEQ ID NO:25, and the VL consists of SEQ ID NO:11. In some embodiments, the present invention provides an antibody, wherein the VH comprises SEQ ID NO:7 and the VL comprises SEQ ID NO:11. In some embodiments, the present invention provides an antibody, wherein the VH comprises SEQ ID NO:17 and the VL comprises SEQ ID NO:11. In some embodiments, the present invention provides an antibody, wherein the VH comprises SEQ ID NO:22 and the VL comprises SEQ ID NO:11. In some embodiments, the present invention provides an antibody, wherein the VH comprises SEQ ID NO:25 and the VL comprises SEQ ID NO:11.

In some embodiments, the present invention provides an antibody wherein the antibody comprises a heavy chain (HC) comprising a sequence selected from SEQ ID NO:1, SEQ ID NO:15, SEQ ID NO:18, SEQ ID NO:3, SEQ ID NO:20, or SEQ ID NO:23, and a light chain (LC) comprising SEQ ID NO:5. In some embodiments, the present invention provides an antibody wherein the antibody comprises a HC comprising a sequence selected from SEQ ID NO:1 or SEQ ID NO:3 and a LC comprising SEQ ID NO:5. In some embodiments, the present invention provides an antibody wherein the antibody comprises a HC comprising SEQ ID NO:15 and a LC comprising SEQ ID NO:5. In some embodiments, the present invention provides an antibody wherein the antibody comprises a HC comprising a sequence selected from SEQ ID NO:18 or SEQ ID NO:20 and a LC comprising SEQ ID NO:5. In some embodiments, the present invention provides an antibody wherein the antibody comprises a HC comprising SEQ ID NO:23 and a LC comprising SEQ ID NO:5.

In some embodiments, the present invention provides an antibody wherein the antibody comprises a heavy chain (HC) comprising amino acids 2-451 selected from SEQ ID NO:1, SEQ ID NO:15, SEQ ID NO:18, SEQ ID NO:3, SEQ ID NO:20, or SEQ ID NO:23, and a light chain (LC) comprising SEQ ID NO:5. In some embodiments, the present invention provides an antibody wherein the antibody comprises a HC comprising amino acids 2-451 selected from SEQ ID NO:1 or SEQ ID NO:3 and a LC comprising SEQ ID NO:5. In some embodiments, the present invention provides an antibody wherein the antibody comprises a HC comprising amino acids 2-451 of SEQ ID NO:15 and a LC comprising SEQ ID NO:5. In some embodiments, the present invention provides an antibody wherein the antibody comprises a HC comprising amino acids 2-451 selected from SEQ ID NO:18 or SEQ ID NO:20 and a LC comprising SEQ ID NO:5. In some embodiments, the present invention provides an antibody wherein the antibody comprises a HC comprising amino acids 2-451 of SEQ ID NO:23 and a LC comprising SEQ ID NO:5.

In some embodiments, the present invention provides an antibody wherein the antibody comprises a HC consisting of a sequence selected from SEQ ID NO:1, SEQ ID NO:15, SEQ ID NO:18, SEQ ID NO:3, SEQ ID NO:20, or SEQ ID NO:23, and a LC consisting of SEQ ID NO:5. In some embodiments, the present invention provides an antibody wherein the antibody comprises a HC consisting of a sequence selected from SEQ ID NO:1 or SEQ ID NO:3 and a LC consisting of SEQ ID NO:5. In some embodiments, the present invention provides an antibody wherein the antibody comprises a HC consisting of SEQ ID NO:15 and a LC consisting of SEQ ID NO:5. In some embodiments, the present invention provides an antibody wherein the antibody comprises a HC consisting of a sequence selected from SEQ ID NO:18 or SEQ ID NO:20 and a LC consisting of SEQ ID NO:5. In some embodiments, the present invention provides an antibody wherein the antibody comprises a HC consisting of a sequence of SEQ ID NO:23 and a LC consisting of SEQ ID NO:5.

In some embodiments, the antibody has an engineered human IgG1 or IgG4 isotype.

Optionally, certain antibodies of the present invention contain an Fc portion which is derived from human IgG1. IgG1 is well known to bind the proteins of the Fc-gamma receptor family (FcγR) as well as C1q. Interaction with these receptors can induce antibody-dependent cell cytotoxicity (ADCC) and complement-dependent cytotoxicity (CDC). Therefore, certain amino acid substitutions may be introduced into IgG1 Fc for certain antibodies of the present invention, including Antibodies IV and V, to ablate immune effector function. Mutations in the CH2 region of the anti-IL-1β portion of the antibody may include positions 234, 235, and 265 (EU numbering).

In some embodiments, the antibody has an engineered human IgG4 isotype. In particular embodiments, the antibodies of the present invention are IgG4 antibodies, and thus contain an IgG4 Fc region, or an Fc region derived from human IgG4, e.g., a modified IgG4 Fc region. According to some embodiments amino acid substitutions are introduced into the IgG4 Fc region. In certain embodiments the antibodies of the present invention are IgG4 antibodies and have modifications in the constant region of both HCs that reduce effector function, including the amino acid alanine at both residues 239 and 240 (residue numbering based on the exemplified HC of SEQ ID NO.18). In certain embodiments the antibodies of the present invention are IgG4 antibodies and have modifications in the constant region of both HCs that reduce effector function including the amino acid alanine at both residues 239 and 240 and have further modifications in the constant region of both HCs promoting stability including the amino acid proline at residue 233 and the deletion of the amino acid lysine at residue 443 (residue numbering based on the exemplified HC of SEQ ID NO.2).

According to some embodiments amino acid substitutions are introduced into the IgG4 Fc region. For example, in some embodiments, a serine to proline mutation is introduced at position 228 (“S228P” according to IMGT or EU numbering), a phenylalanine to alanine mutation is introduced at position 234 (“F234A” according to IMGT or EU numbering), and/or a leucine to alanine mutation is introduced at position 235 (“L235A” according to IMGT or EU numbering). According to some embodiments of antibodies of the present invention, the Fc region comprises S228P, F234A and L235A (according to EU Index numbering). In some embodiments, the present invention provides nucleic acid sequence encoding SEQ ID NO:1, SEQ ID NO:15, SEQ ID NO:18, SEQ ID NO:3, SEQ ID NO:20, or SEQ ID NO:23, and a second nucleic acid encoding SEQ ID NO:5.

Another embodiment is a vector comprising a first nucleic acid sequence encoding SEQ ID NO:1, SEQ ID NO:15, SEQ ID NO:18, SEQ ID NO:3, SEQ ID NO:20, or SEQ ID NO:23, and a second nucleic acid sequence encoding SEQ ID NO:5. In other embodiments, the nucleic acids are present on separate vectors. Another embodiment is a first vector comprising a nucleic acid sequence encoding SEQ ID NO:1, SEQ ID NO:15, SEQ ID NO:18, SEQ ID NO:3, SEQ ID NO:20, or SEQ ID NO:23, and a second vector comprising a nucleic acid sequence encoding SEQ ID NO:5.

In some embodiments, the present invention provides a cell comprising the vectors described herein. In one embodiment, the present disclosure provides a cell with a first nucleic acid sequence encoding SEQ ID NO:1, SEQ ID NO:15, SEQ ID NO:18, SEQ ID NO:3, SEQ ID NO:20, or SEQ ID NO:23, and a second nucleic acid sequence encoding SEQ ID NO:5. In another embodiment, the present invention provides a cell comprising a first vector comprising a nucleic acid sequence encoding SEQ ID NO:1, SEQ ID NO:15, SEQ ID NO:18, SEQ ID NO:3, SEQ ID NO:20, or SEQ ID NO:23, and a second vector comprising a nucleic acid sequence encoding SEQ ID NO:5. In an embodiment, the cell is a mammalian cell.

In an embodiment, the present invention provides an antibody comprising two light chains and two heavy chains, wherein each light chain has the amino acid sequence given in SEQ ID NO:5 and each heavy chain has the amino acid sequence given in SEQ ID NO:1.

In an embodiment, the present invention provides an antibody comprising two light chains and two heavy chains, wherein each light chain has the amino acid sequence given in SEQ ID NO:5 and each heavy chain has the amino acid sequence given in SEQ ID NO:15.

In an embodiment, the present invention provides an antibody comprising two light chains and two heavy chains, wherein each light chain has the amino acid sequence given in SEQ ID NO:5 and each heavy chain has the amino acid sequence given in SEQ ID NO:18. In an embodiment, the present invention provides an antibody comprising two light chains and two heavy chains, wherein each light chain has the amino acid sequence given in SEQ ID NO:5 and each heavy chain has the amino acid sequence given in SEQ ID NO:3. In an embodiment, the present invention provides an antibody comprising two light chains and two heavy chains, wherein each light chain has the amino acid sequence given in SEQ ID NO:5 and each heavy chain has the amino acid sequence given in SEQ ID NO:20. In an embodiment, the present invention provides an antibody comprising two light chains and two heavy chains, wherein each light chain has the amino acid sequence given in SQ ID NO:5 and each heavy chain has the amino acid sequence given in SEQ ID NO:23.

In an embodiment, the present invention provides a process of producing an antibody comprising culturing the cell under conditions such that the antibody is expressed and recovering the expressed antibody from the culture medium. In another embodiment, the present invention provides an antibody produced by culturing the cell under conditions such that the antibody is expressed and recovering the expressed antibody from the culture medium. Processes for expressing an antibody or culturing cells under conditions such that an antibody is expressed are well known in the art, as are processes for recovering the antibody.

In an embodiment, the present invention provides a pharmaceutical composition comprising the antibody and a pharmaceutically acceptable excipient, diluent or carrier.

In an embodiment, the present invention provides a method of treating disease comprising administering the antibody of the present invention, and an acceptable carrier, diluent, or excipient.

In an embodiment, the present invention provides a method of preventing disease, comprising administering the antibody of the present invention and an acceptable carrier, diluent, or excipient.

In a further embodiment, the present invention provides a method of treating diseases, wherein the disease is inflammatory disease.

In a further embodiment, the present invention provides a method of preventing diseases, wherein the disease is inflammatory disease.

In an embodiment, the present invention provides an antibody of the present invention, for use in therapy. In an embodiment, the present invention provides an antibody of the present invention, for use in the treatment of inflammatory disease. In a further embodiment, the present invention provides an antibody of the present invention, for use in the treatment of an inflammatory disease, wherein the inflammatory disease is cardiovascular disease. In a further embodiment, the present invention provides an antibody of the present invention, for use in the treatment of an inflammatory disease, wherein the inflammatory disease is cancer.

In a further embodiment, the present invention provides the use of an antibody of the present invention for the manufacture of a medicament for the treatment of cardiovascular disease or cancer. In a further embodiment, the present invention provides the use of an antibody of the present invention that binds to and antagonizes protein IL-1beta, for use in the treatment of inflammatory disease.

As used herein, an “antibody” is an immunoglobulin (IgG) molecule that binds antigen. A full-length antibody as it exists naturally is an IgG molecule comprising 2 heavy (H) chains and 2 light (L) chains interconnected by disulfide bonds. The amino terminal portion of each chain includes a variable region of about 100-110 amino acids primarily responsible for antigen recognition via the complementarity determining regions (CDRs) contained therein. The carboxy-terminal portion of each chain defines a constant region primarily responsible for effector function.

The CDRs are interspersed with regions that are more conserved, termed framework regions (FR). Each light chain variable region (LCVR, also known as VL) and heavy chain variable region (HCVR, also known as VH) is composed of 3 CDRS and 4 FRs, arranged from amino-terminus to carboxy-terminus in the following order: FR1, CDR1, FR2, CDR2, FR3, CDR3, FR4. The 3 CDRs of the light chain are referred to as “LCDR1, LCDR2, and LCDR3” and the 3 CDRs of the heavy chain are referred to as “HCDR1, HCDR2, and HCDR3.” The CDRs contain most of the residues which form specific interactions with the antigen. The numbering and positioning of CDR amino acid residues within the VL and VH regions in accordance with the well-known North numbering convention.

Light chains are classified as kappa or lambda and are characterized by a particular constant region known in the art. Heavy chains are classified as gamma, mu, alpha, delta, or epsilon, and define the isotype of an antibody as IgG, IgM, IgA, IgD, or IgE, respectively. IgG antibodies can be further divided into subclasses, e.g., IgG1, IgG2, IgG3, IgG4. Each heavy chain type is characterized by a particular constant region with a sequence well known in the art.

In some biological systems and processes for producing, antibodies may undergo co- and post-translational modifications, such as glycosylation, deamidation, acylation, oxidation, cyclization, fucosylation, among other modifications that are well known in the art. Another known modification is cyclization of glutamine or glutamate to pyroglutamate (often abbreviated pyrGlu, pyrE, pGlu, or pE) at the N-terminus of the heavy chain variable region that comprises the heavy chain. Depending on the methods and antibodies used, the percentage of glutamate that is converted to pyroglutamate varies, and may represent a mixture, involve substantially all antibodies being produced, or a very low percentage of the antibodies.

As used herein, the term “monoclonal antibody” (mAb) refers to an antibody that is derived from a single copy or clone including, for example, any eukaryotic, prokaryotic, or phage clone, and not the method by which it is produced. Monoclonal antibodies of the present invention preferably exist in a homogenous or substantially homogenous population. Complete mAbs contain 2 heavy chains and 2 light chains. Monoclonal antibodies can be produced, for example, by hybridoma technologies, recombinant technologies, phage display technologies, synthetic technologies, e.g., CDR-grafting, or combinations of such or other technologies known in the art.

Protein “IL-1β” (also known as IL-1 beta or IL-1β or Interleukin-1ß have the same meaning herein) refers to the primary circulating form of IL-1. It is produced as a pre-cursor (pro-IL-1-Beta) that is activated via the NLRP3 inflammasome under a variety of inflammatory diseases.

“Inflammatory” as used herein includes both inflammatory and autoinflammatory diseases. The term “inflammatory diseases” includes, but is not limited to, ASCVD, heart failure, cancer, and rare inherited disorders due to overproduction of IL-1β.

The term “treating” (or “treat” or “treatment”) refers to processes involving a slowing, interrupting, arresting, controlling, stopping, reducing, or reversing the progression or severity of a symptom, disorder, condition, or disease, but does not necessarily involve a total elimination of all disease-related symptoms, conditions, or disorders associated with IL-1-beta activity. The term “preventing” (or “prevent”) refers to keep something from happening, existing, or occurring and/or to hinder or stop from doing something.

The term “cardiovascular disease” herein refers to a class of diseases that involves the heart or blood vessels. Common manifestations of CVD include, but are not limited to: MI, stroke, ASCVD, heart failure, and arrythmia.

Engineered human antibodies in addition to those disclosed herein exhibiting similar functional properties according to the present invention can be generated using several different methods. The specific antibody compounds disclosed herein can be used as templates or parent antibody compounds to prepare additional antibody compounds. In one approach, the parent antibody compound CDRs are grafted into a human framework that has a high sequence identity with the parent antibody compound framework. The sequence identity of the new framework will generally be at least about 80%, at least about 85%, at least about 90%, at least about 95%, or at least about 99% identical to the sequence of the corresponding framework in the parent antibody compound. This grafting may result in a reduction in binding affinity compared to that of the parent antibody. If this is the case, the framework can be back-mutated to the parent framework at certain positions based on specific criteria disclosed by Queen et al. Al (1991) Proc. Natl. Acad. Sci US 88:2869. Additional references describing methods useful in humanizing antibodies include U.S. Pat. Nos. 4,816,397; 5,225,539, and 5,693,761; computer programs ABMOD and ENCAD as described in Levitt (1983) J. Mol. Biol. 168:595-620; and the method of Winter and co-workers (Jones et Al. (1986) Nature 321:522-525; Riechmann et al. (1988) Nature 332:323-327; and Verhoeyen et al. (1988) Science 239:1534-1536.

The identification of residues to consider for back-mutation can be carried out as follows:
When an amino acid falls under a category in which the amino acid in the human framework region of the acceptor framework is unusual for human frameworks at that position, whereas the corresponding amino acid in the donor immunoglobulin is typical for human frameworks at that position, the framework amino acid of the human germ-line sequence that is being used (the “acceptor framework”) is replaced by a framework amino acid from a framework of the parent antibody compound (the “donor framework”).

When each of the amino acids in the human framework region of the acceptor framework and a corresponding amino acid in the donor framework is generally unusual for human frameworks at that position, such amino acid can be replaced by an amino acid typical for human frameworks at that position. This back-mutation criterion enables one to recover the activity of the parent antibody compound.

Another approach to generating engineered human antibodies exhibiting similar functional properties to the antibody compounds disclosed herein involves randomly mutating amino acids within the grafted CDRs without changing the framework and screening the resultant molecules for binding affinity and other functional properties that are as good as, or better than, those of the parent antibody compounds. Single mutations can also be introduced at each amino acid position within each CDR, following by assessing the effects of such mutations on binding affinity and other functional properties. Single mutations producing improved properties can be combined to assess their effects in combination with one another.

Further, a combination of both foregoing approaches is possible. After CDR grafting, one can back-mutate specific framework regions in addition to introducing amino acid changes in the CDRs. This methodology is described in Wu et al., (1999) J. Mol. Biol. 294:151-162.

The engineered human antibodies of the present invention can be used as medicaments in human medicine, administered by a variety of routes. Most preferably, such compositions are for parenteral administration. Such pharmaceutical compositions can be prepared by methods well known in the art (See, e.g., Remington: The Science and Practice of Pharmacy; 19^{th} ed. (1995), A. Gennaro et al., Mack Publishing Co.) and comprise an engineered human antibody as disclosed herein, and a pharmaceutically acceptable carrier, diluent, or excipient.

The results of the following assays demonstrate that the exemplified monoclonal antibodies and antigen-binding fragments thereof of the present invention bind IL-1β and/or neutralize and therefore may be used for treating inflammatory diseases such as cardiovascular disease or cancer.

### EXAMPLE 1: ANTIBODY EXPRESSION AND PURIFICATION

Significant problems with chemical and physical stability were encountered when constructing an IL-1β antibody of the present invention. For example, problems encountered included, among other things, low binding affinity, variable region deamidation, oxidation, and low potency.

Modifications were therefore engineered to improve the binding affinity and improve chemical and physical stability of the antibodies. Amino acid modifications were introduced throughout both the heavy and light chains. The antibodies of the present invention include multiple residue changes from original constructs, are identified as possessing high binding affinity and being chemically and physically stable. None of the modifications comprising the antibody of the present invention were identified in the initial construct.

Exemplified anti-IL-1β antibodies of the present invention are presented in Table 1.

Antibodies I-VI can be made and purified as follows. An appropriate host cell, such as HEK 293 or CHO, is either transiently transfected with an expression system for secreting antibodies using an optimal predetermined HC: LC vector ratio encoding sequences of Antibody I_HC, Antibody II_HC, Antibody III_HC, Antibody IV_HC, Antibody V_HC, Antibody VI_HC, and a common LC sequence. Clarified media, into which the antibody has been secreted, is purified using any of many commonly used techniques. For example, the medium may be conveniently applied to a Protein A or G column that has been equilibrated with a compatible buffer, such as phosphate buffered saline (pH 7.4). The column is washed to remove nonspecific binding components. The bound antibody is eluted, for example, by pH gradient (such as 0.1 M sodium phosphate buffer pH 6.8 to 0.1 M sodium citrate buffer pH 2.5). Antibody fractions are detected, such as by SDS-PAGE, and then are pooled. Further purification is optional, depending on the intended use. The antibody may be concentrated and/or sterile filtered using common techniques. Soluble aggregate and multimers may be effectively removed by common techniques, including size exclusion, hydrophobic interaction, or ion exchange chromatography. The purity of the antibody after these chromatography steps is greater than 99%. The product may be immediately frozen at −70° C. or may be lyophilized or preserved in 4° C. for immediate use.

### EXAMPLE 2: ANTIBODY DISCOVERY AND ENGINEERING

Four in-house phage libraries are used to pan against human (hu) IL-1β in solution panning for three rounds. Phage plaques are randomly picked and evaluated for huIL-1β binding activity. After sequencing and binding confirmation, a panel of anti-huIL-1β phage hits are cloned into and expressed as human IgG.

To increase antibody affinity, mutations are introduced into all individual residues of heavy chain complementarity determining regions (HCDRs) of parental IL-1β phage antibody. From filter-lift screening, beneficial mutations are selected to make a combinatorial library. After filter-lift and binding titration, individual combinatorial clones are sequenced, and binding characteristics are determined. Additionally, framework (FW) substitutions were made to the light chain to revert FW1, FW2, FW3 and FW4 sequences to its germline state in order to reduce potential immunogenicity and post-translational modifications (PTM).

Engineered and/or optimized anti-IL-1β antibodies referred to herein as Antibody I, Antibody II, Antibody III, Antibody IV, Antibody V, and Antibody VI are obtained, having the amino acid sequences of the variable regions of the heavy chain and light chain, and the complete heavy chain and light chain amino acid sequences, and the nucleotide sequences encoding the same, as listed in the section entitled “Amino Acid and Nucleotide Sequences”. The sequence IDs corresponding to these fragments are shown below in Table 1, as well as the light chain and heavy chain CDR amino acid sequences.

TABLE 1

| | Antibody I | Antibody II | Antibody III | Antibody IV | Antibody V | Antibody VI |
|---|---|---|---|---|---|---|
| Protein | SEQ ID NO | SEQ ID NO | SEQ ID NO | SEQ ID NO | SEQ ID NO | SEQ ID NO |
| HC | 1 | 15 | 18 | 3 | 20 | 23 |
| HCVR | 7 | 17 | 22 | 7 | 22 | 25 |
| LC | 5 | 5 | 5 | 5 | 5 | 5 |
| LCVR | 11 | 11 | 11 | 11 | 11 | 11 |
| HCDR1 | 8 (X₁ = | 8 (X₁ = | 8 (X₁ = | 8 (X₁ = | 8 (X₁ = | 8 (X₁ = |
| | F) | L) | L) | F) | L) | F) |
| HCDR2 | 9 (X₂ = | 9 (X₂ = | 9 (X₂ = | 9 (X₂ = | 9 (X₂ = | 9 (X₂ = |
| | P) | D) | D) | P) | D) | D) |
| HCDR3 | 10 (X₃ = | 10 (X₃ = | 10 (X₃ = | 10 (X₃ = | 10 (X₃ = | 10 (X₃ = |
| | T) | P) | T) | T) | T) | T) |
| LCDR1 | 12 | 12 | 12 | 12 | 12 | 12 |
| LCDR2 | 13 | 13 | 13 | 13 | 13 | 13 |
| LCDR3 | 14 | 14 | 14 | 14 | 14 | 14 |

### EXAMPLE 3: NEUTRALIZATION OF HUMAN OR CYNO IL-18 IN VITRO

Recombinant human or cyno IL-1β is produced in E. coli as N-terminal HIS-SUMO fusion protein. The protein is purified using HisPur Ni-NTA chromatography and followed by endotoxin removal. The purified fusion protein is then treated with SUMO protease Ulp1 to cleave the HIS-SUMO off the fusion protein. The cleaved HIS-SUMO protein is removed from the reaction by HisPur Ni-NTA, and the untagged IL-1β was further purified to homogeneity using Superdex 75 size-exclusion chromatography.

Antibodies of the present invention are expected to neutralize IL-1β. Neutralization of IL-1β activity by antibodies of the present invention may be assessed by one or more IL-1β cell-based activity assays, for example, as described below.

Screening for neutralizers of IL-1β/IL-1R binding may initially be done through a high-throughput cell-based assay using HeLa cells expressing a Luciferase gene under control of a NF-kB promoter. This assay uses NF-κB-Luciferase reporter signal as a readout of recombinant IL-1β induced signaling. Neutralization of IL-1β can then be quantified by measuring the level of reduction of Luciferase activity upon titration of anti-IL-1β antibodies. Alternatively, another in vitro neutralization assay such as a HEK-Blue cell-based assay is described in detail below.

HEK-Blue™ IL-1β cells (InvivoGen Cat. #hkb-il1b) expressing an NF-κB/AP-1-inducible SEAP (Secreted embryome alkaline phosphatase) reporter gene are used to monitor activity of IL-1β. Specifically, HEK-Blue™ IL-1β cells are cultured in a T-75 flask in growth medium (DMEM, 4.5 g/L glucose, 2 mM L-Glutamine, 10% (v/v) fetal bovine serum, 50 U/mL penicillin, 50 μg/mL streptomycin, 100 μg/mL Normocin™, 100 μg/mL Zeocin™ and 200 μg/mL Hygromycin B Gold) until 90% confluence. Cells are washed with PBS (without Ca⁺⁺ and Mg⁺⁺) twice and incubated in 1 mL of PBS for 2 minutes. Cells are then detached by patting on the side of the flask, resuspended with 10 mL test medium (DMEM, 4.5 g/L glucose, 2 mM L-Glutamine, 10% (v/v) heat-inactivated FBS (30 min at 56° C.), 50 U/mL penicillin, 50 μg/mL streptomycin, 100 μg/mL Normocin™), counted and diluted to 0.33×10⁶ cells/mL with test medium. Recombinant human or cyno IL-1β and test articles are prepared to the desired concentrations in test medium. 40 L of antibody (5x concentration) is mixed with 10 μL of IL-1β (20× concentration, final concentration in assay was 4 pM) in a BioCoat poly-D-lysine plate (Corning 354461) and incubated at room temperature for 30 minutes. 150 μL of HEK-Blue™ IL-1β cell suspension at 0.33×10⁶ cells/mL is dispensed to each well of the poly-D-lysine plate containing antibody and IL-1β mixture. The plate is incubated at 37° C., 5% CO₂ and 90% relative humidity overnight. On the second day, 25 μL culture medium from the poly-D-lysine plate is transferred to a Costar assay plate (Corning 3695). 75 μL of QUANTI-Blue detection solution (Invivogen Catalog #rep-qb1, rep-qb2) pre-warmed to 37° C. is added to the assay plate. The assay plate is covered and incubated at 37° C. for 1 hour before reading on a plate reader (SpectraMax Plus, Molecular Device) at OD₆₅₀ nm. Data is normalized and expressed as percent inhibition of 4 pM IL-1β: 0% Inhibition=4 PM IL-1β, 100% Inhibition=0 pM IL-1β. Neutralizing anti-hIL-1β antibodies block the activity recombinant human IL-1β to stimulate HEK-Blue™ IL-1β cells. Relative potencies of the neutralizing antibodies are calculated using 4-parameter logistic fit and expressed in IC₅₀ values.

Activities of the antibodies of the present invention (Antibodies I-VI) for neutralization of human or cyno IL-1β are summarized in Table 2. Canakinumab is included as a comparator. Antibodies I-VI demonstrate comparable or greater relative potencies (up to 3-fold, as indicated by the IC50 values) compared to Canakinumab for neutralization of human IL-1β. Antibodies I-VI are also active in neutralizing cyno IL-1β, while Canakinumab does not show neutralizing activity for cyno IL-1β in this assay.

TABLE 2
Neutralization of human or cyno IL-1β in vitro

| | Neutralization of human IL-1β | | | Neutralization of cyno IL-1β | | |
|---|---|---|---|---|---|---|
| Molecules | Avg. IC50 (pM) | SD (pM) | N | Avg. IC50 (pM) | SD (pM) | “n” |
| Canakinumab | 25.99 | 3.71 | 8 | Not active* | na | 4 |
| Antibody I | 8.48 | 1.56 | 4 | 21.83 | 6.42 | 3 |
| Antibody II | 19.00 | 1.96 | 3 | 69.84 | 10.12 | 2 |
| Antibody III | 21.44 | 3.24 | 4 | 54.82 | 1.02 | 3 |
| Antibody IV | 9.04 | na | 1 | 30.05 | na | 1 |
| Antibody V | 27.79 | na | 1 | 112.70 | na | 1 |
| Antibody VI | 15.94 | na | 1 | 53.44 | na | 1 |

“n” represents number of independent experiments from different dates. Within each experiment, there are typically 3 replicates.
Not active* is defined as at the highest antibody concentration tested in the assay, the inhibition of IL-1β signal is lower than the standard deviation.
na: not applicable

### EXAMPLE 4: NEUTRALIZATION OF HUMAN IL-18 IN VIVO

Human IL-1β can bind to and stimulate the mouse IL-1 receptor, leading to an elevation of mouse cytokine IL-6. For testing in vivo neutralizing activities of the antibodies of the present invention, an optimized protocol is described below. Specifically, male C57BL/6 mice from Envigo are used for the study at approximately 9 weeks of age. Mice are fed a normal chow diet (Harlan Teklad diet, 2014) and randomized to treatment groups by body weight (n=5-8/group). Antibodies of the present invention and control antibodies are dissolved in saline and administered subcutaneously at dose levels as indicated. Twenty-four hours later, human IL-1β dissolved in saline is dosed intraperitoneally at 1 μg/kg dose level. Two hours later, blood samples are collected via retro orbital bleeding followed by centrifugation at 2000 g for 3 minutes to isolate serum samples.

Mouse IL-6 levels in serum are determined using the V-PLEX Mouse IL-6 kit (Meso Scale Discovery, Cat #K152QXD-2) following the manufacturer's instructions. Briefly, an MSD plate is washed 3 times with 150 μL Wash buffer. 50 μL of previously prepared calibrators (serial dilution), control and test samples (1:10 dilution) are transferred to appropriate wells on the plate, followed by 2 hours of shaking (500˜1000 rpm) at room temperature. The plate is washed 3 times with 150 μL Wash buffer. 25 ul of Detection Antibody solution is then added to each well, followed by 2 hours of shaking (500˜1000 rpm) at room temperature. The plate is washed 3 times with 150 μL Wash buffer. 150 μL of 2x Read Buffer is added to each well. The plate is immediately read on an MSD SQ120 plate reader. IL-6 concentrations of the test samples are analyzed from the calibration curve using a 4-parameter logistic fit.

Isotype matched control antibody (IgG4-PAA) is used as negative control for the study. Data are calculated as percent inhibition compared to mean IL-6 level of the control group. Statistical significance for the difference of means are assessed using one-way ANOVA, Dunnett's post-hoc with JMP11 software. Antibodies of the present invention (Antibodies I-III) dose dependently block the effects of human IL-1β to stimulate the mouse IL-1 receptor-mediated increase of mouse IL-6 in a manner that is comparable to Canakinumab (Tables 3-5). Antibody II show greater activities compared to Canakinumab at the same dose levels, consistent with its higher relative potency in neutralizing human IL-1β in vitro.

TABLE 3
Neutralization of human IL-1β in vivo using Antibody II

| | | % change vs | |
|---|---|---|---|
| | Dose level | IgG4-PAA | SE |
| Molecules | (μg/kg) | control | (of % change) |
| IgG4-PAA | 4000 | 0.0 | 12.2 |
| control | | | |
| Canakinumab | 60 | 14.9 | 10.3 |
| Canakinumab | 200 | 10.2 | 15.9 |
| Canakinumab | 600 | −54.6 | 11.3 |
| Canakinumab | 2000 | −86.6 | 2.8 |
| Canakinumab | 4000 | −95.3 | 1.2 |
| Antibody II | 60 | −52.2 | 4.1 |
| Antibody II | 200 | −64.2 | 5.6 |
| Antibody II | 600 | −83.0 | 1.3 |
| Antibody II | 2000 | −90.5 | 1.2 |
| Antibody II | 4000 | −91.5 | 1.1 |

SE = standard error

TABLE 4
Neutralization of human IL-1β in vivo using Antibody I

| | | % change vs | |
|---|---|---|---|
| | Dose level | IgG4-PAA | SE |
| Molecules | (μg/kg) | control | (of % change) |
| IgG4-PAA | 4000 | 0.0 | 10.3 |
| control | | | |
| Canakinumab | 60 | −5.2 | 11.9 |
| Canakinumab | 200 | −36.9 | 7.9 |
| Canakinumab | 600 | −75.7 | 3.2 |
| Canakinumab | 2000 | −91.8 | 2.4 |
| Canakinumab | 4000 | −96.1 | 0.4 |
| Antibody I | 60 | −86.7 | 2.8 |
| Antibody I | 200 | −89.3 | 4.5 |
| Antibody I | 600 | −97.4 | 0.5 |
| Antibody I | 2000 | −97.0 | 0.5 |
| Antibody I | 4000 | −98.0 | 0.2 |

TABLE 5
Neutralization of human IL-1β in vivo using Antibody III

| | | % change vs | |
|---|---|---|---|
| | Dose level | IgG4-PAA | SE |
| Molecules | (μg/kg) | control | (of % change) |
| IgG4-PAA | 4000 | 0.0 | 10.3 |
| control | | | |
| Canakinumab | 60 | −5.2 | 11.9 |
| Canakinumab | 200 | −36.9 | 7.9 |
| Canakinumab | 600 | −75.7 | 3.2 |
| Canakinumab | 2000 | −91.8 | 2.4 |
| Canakinumab | 4000 | −96.1 | 0.4 |
| Antibody III | 60 | −36.4 | 6.6 |
| Antibody III | 200 | −55.5 | 6.2 |
| Antibody III | 600 | −77.9 | 3.7 |
| Antibody III | 2000 | −90.8 | 3.3 |
| Antibody III | 4000 | −96.2 | 0.3 |

### EXAMPLE 5: BINDING AFFINITY MEASUREMENT OF ANTIBODY I, ANTIBODY II, ANTIBODY III AND BY MSD-SET

MSD (Meso Scale Discovery) electrochemiluminescence assay is utilized to measure the affinity of Antibody I, Antibody II, Antibody III, and Canakinumab to human IL-1β. First, the equilibrium mixture of antibodies and human IL-1β is set up; in the mixture the antibody concentrations are kept constant at 1 pM, 10 pM, and 100 pM whereas the ligand is titrated in the concentration ranging from 0.9 nM to 0.00004 nM (2.5 times dilution between concentrations). The equilibrium mixture is set up in sealed non-binding 96-well plate at 37° C. for 72 hours.

MSD Gold Streptavidin plates are used to detect the free antibodies in the equilibrium mixture. The MSD plates are first blocked with blocking buffer (PBS+1% BSA) for 1 hour on a shaker set at 800 rpm and then washed 3 times with washing buffer PBST (PBS+0.05% Tween 20). The plates are coated with biotinylated human IL-1β followed by 3× washing with PBST. The equilibrium mixtures are added to the coated plate, incubated at room temperature with shaking for 2.5 mins and immediately washed 3× with PBST. The goat anti-human Sulfo-TAG antibody is added to the plate and incubated at room temperature for 1 hour with shaking. After three more washes, MSD Read Buffer at 1:2 concentration diluted in MilliQ water is added to the wells. Immediately afterwards, the plates are read using an MSD Sector Imager SI6000 instrument.

For data evaluation, the readout of the MSD instrument is imported into a customized Excel or GraphPad Prism 8-based evaluation program, which automatically plots the titration data, and calculates the KD values as well as statistical parameters.

Antibodies of the present invention (Antibodies I-III) demonstrate similar binding affinities for human IL-1β compared to Canakinumab (Table 6).

TABLE 6
Binding affinities for the antibodies (Ligand: Human IL-1β @ 37° C.)

| | Affinity |
|---|---|
| Test Compound | (K_{D} ± SE) pM |
| Antibody I | 14.7 ± 0.1 |
| Antibody II | 31.6 ± 0.6 |
| Antibody III | 24.5 ± 0.3 |
| Canakinumab | 13.5 ± 0.2 |

Amino Acid and Nucleotide Sequences

SEQ ID NO: 1 (HC of Antibody I)
X₄VQLLESGGGLVQPGGSLRLSCAASGFTFSSYSFSWVRQAPGKGLEWVSAISVS
GGSTYYAPSVKGRFTISRDNSKNTLYLQMNSLRAEDTAVYYCARDDLITTRGTFY
NWFDPWGQGTLVTVSSASTKGPSVFPLAPCSRSTSESTAALGCLVKDYFPEPVTV
SWNSGALTSGVHTFPAVLQSSGLYSLSSVVTVPSSSLGTKTYTCNVDHKPSNTKV
DKRVESKYGPPCPPCPAPEAAGGPSVFLFPPKPKDTLMISRTPEVTCVVVDVSQED
PEVQFNWYVDGVEVHNAKTKPREEQFNSTYRVVSVLTVLHQDWLNGKEYKCK
VSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEAL
HNHYTQKSLSLSLG
Wherein X₄ is E or pE
SEQ ID NO: 2 (HC DNA of Antibody I)
gaggtgcagctgttggagtctgggggaggcttggtacagcctggggggtccctgagactctcctgtgcagcctctggattcacct
ttagcagctattccttcagctgggtccgccaggctccagggaaggggctggagtgggtctcagctattagtgtcagtggtggtag
cacatactacgcaccctccgtgaagggtcggttcaccatctccagagacaattccaagaacacgctgtatctgcaaatgaacagc
ctgagagccgaggacacggccgtatattactgtgcgagagatgacctaataacaacgcgtggtaccttctacaactggttcgacc
cctggggccagggaaccctggtcaccgtctcctcagccagcaccaagggcccatcggtcttcccactagcgccctgctccagg
agcacctccgagagcacagccgccctgggctgcctggtcaaggactacttccccgagccggtgacggtgtcgtggaactcag
gagccctgaccagcggcgtgcacaccttcccggctgtcctacagtcctcaggactctactccctcagcagcgtggtgaccgtgc
cctccagcagcttgggcacgaagacctacacctgcaacgtagatcacaagcccagcaacaccaaggtggacaagagagttga
gtccaaatatggtcccccatgcccaccctgcccagcacctgaggccgccgggggaccatcagtcttcctgttccccccaaaacc
caaggacactctcatgatctcccggacccctgaggtaacgtgcgtggtggtggacgtgagccaggaagaccccgaggtccagt
tcaactggtacgtggatggcgtggaggtgcataatgccaagacaaagccgcgggaggagcagttcaacagcacgtaccgtgt
ggtcagcgtcctcaccgtcctgcaccaggactggctgaacggcaaggagtacaagtgcaaggtctccaacaaaggcctcccgt
cctccatcgagaaaaccatctccaaagccaaagggcagccccgagagccacaggtgtacaccctgcccccatcccaggagga
gatgaccaagaaccaggtcagcctgacctgcctggtcaaaggcttctaccccagcgacatcgccgtggagtgggaaagcaatg
ggcagccggagaacaactacaagaccacgcctcccgtgctggactccgacggctccttcttcctctacagcaggctaaccgtg
gacaagagcaggtggcaggaggggaatgtcttctcatgctccgtgatgcatgaggctctgcacaaccactacacacagaagag
cctctccctgtctctgggt
SEQ ID NO: 3
(HC of Antibody IV)
X₄VQLLESGGGLVQPGGSLRLSCAASGFTFSSYSFSWVRQAPGKGLEWVSAISVS
GGSTYYAPSVKGRFTISRDNSKNTLYLQMNSLRAEDTAVYYCARDDLITTRGTFY
NWFDPWGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKV
DKRVEPKSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVMH
EALHNHYTQKSLSLSPGK
Wherein X₄ is E or pE
SEQ ID NO: 4 (HC DNA of Antibody IV)
gaggtgcagctgttggagtctgggggaggcttggtacagcctggggggtccctgagactctcctgtgcagcctctggattcacct
ttagcagctattccttcagctgggtccgccaggctccagggaaggggctggagtgggtctcagctattagtgtcagtggtggtag
cacatactacgcaccctccgtgaagggtcggttcaccatctccagagacaattccaagaacacgctgtatctgcaaatgaacagc
ctgagagccgaggacacggccgtatattactgtgcgagagatgacctaataacaacgcgtggtaccttctacaactggttcgacc
cctggggccagggaaccctggtcaccgtctcctcagctagcaccaagggcccatcggtcttccccctggcaccctcctccaaga
gcacctctgggggcacagcggccctgggctgcctggtcaaggactacttccccgaaccggtgacggtgtcgtggaactcagg
cgccctgaccagcggcgtgcacaccttcccggctgtcctacagtcctcaggactctactccctcagcagcgtggtgaccgtgcc
ctccagcagcttgggcacccagacctacatctgcaacgtgaatcacaagcccagcaacaccaaggtggacaagagagttgag
cccaaatcttgtgacaaaactcacacatgcccaccgtgcccagcacctgaagccgccgggggaccgtcagtcttcctcttcccc
ccaaaacccaaggacaccctcatgatctcccggacccctgaggtcacatgcgtggtggtggacgtgagccacgaagaccctga
ggtcaagttcaactggtatgtggacggcgtggaggtgcataatgccaagacaaagccgcgggaggagcagtacaacagcacg
taccgtgtggtcagcgtcctcaccgtcctgcaccaagactggctgaatggcaaggagtacaagtgcaaggtctccaacaaagcc
ctcccagcccccatcgagaaaaccatctccaaagccaaagggcagccccgagaaccacaggtgtacaccctgcccccatccc
gggaggagatgaccaagaaccaagtcagcctgacctgcctggtcaaaggcttctatcccagcgacatcgccgtggagtggga
gagcaatgggcagccggagaacaactacaagaccacgcctcccgtgctggactccgacggctccttcttcctctattccaagct
caccgtggacaagagcaggtggcagcaggggaacgtcttctcatgctccgtgatgcatgaggctctgcacaaccactacacgc
agaagagcctctccctgtctccgggcaaa
SEQ ID NO: 5 (LC (for Antibody I, Antibody II, Antibody III, Antibody IV, Antibody V,
Antibody VI))
DIQMTQSPSSLSASVGDRVTITCRASQSISSNLNWYQQKPGKAPKLLIYAASSLQS
GVPSRFSGSGSGTDFTLTISSLQPEDFATYYCQQSYSIPWTFGQGTKVEIKRTVAA
PSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
SEQ ID NO: 6 (LCDNA of Antibody I, Antibody II, Antibody III, Antibody IV,
Antibody V, Antibody VI)
gacatccagatgacccagtctccatcctccctgtctgcatctgtaggagacagagtcaccatcacttgccgggcaagtcagagca
ttagcagcaatttaaattggtatcagcagaaaccagggaaagcccctaagctcctgatctatgctgcatccagtttgcaaagtggg
gtcccatcaaggttcagtggcagtggatctgggacagatttcactctcaccatcagcagtctgcaacctgaagattttgcaacttac
tactgtcaacagagttacagtatcccttggacgttcggccaagggaccaaggtggaaatcaaacgaaccgtggctgcaccatct
gtcttcatcttcccgccatctgatgagcagttgaaatctggaactgcctctgttgtgtgcctgctgaataacttctatcccagagagg
ccaaagtacagtggaaggtggataacgccctccaatcgggtaactcccaggagagtgtcacagagcaggacagcaaggaca
gcacctacagcctcagcagcaccctgacgctgagcaaagcagactacgagaaacacaaagtctacgcctgcgaagtcaccca
tcagggcctgagctcgcccgtcacaaagagcttcaacaggggagagtgc
SEQ ID NO: 7 (VH of Antibody I, Antibody IV)
X₄VQLLESGGGLVQPGGSLRLSCAASGFTFSSYSFSWVRQAPGKGLEWVSAISVS
GGSTYYAPSVKGRFTISRDNSKNTLYLQMNSLRAEDTAVYYCARDDLITTRGTFY
NWFDPWGQGTLVTVSS
Wherein X₄ is E or pE
SEQ ID NO: 8 (HCDR1 of Antibody I, Antibody II, Antibody III, Antibody IV, Antibody
V, Antibody VI)
AASGFTFSSYSX₁S
wherein X₁ is F or L
SEQ ID NO: 9 (HCDR2 of Antibody I, Antibody II, Antibody III, Antibody IV, Antibody
V, Antibody VI)
AISVSGGSTYYAX₂SVKG
wherein X₂ is P or D
SEQ ID NO: 10
(HCDR3 of Antibody I, Antibody II, Antibody III, Antibody IV,
Antibody V, Antibody VI)
ARDDLIX₃TRGTFYNWFDP
wherein X₃ is T or P
SEQ ID NO: 11
(VL of Antibody I, Antibody II, Antibody III, Antibody IV,
Antibody V, Antibody VI)
DIQMTQSPSSLSASVGDRVTITCRASQSISSNLNWYQQKPGKAPKLLIYAASSLQS
GVPSRFSGSGSGTDFTLTISSLQPEDFATYYCQQSYSIPWTFGQGTKVEIK
SEQ ID NO: 12
(LCDR1 of Antibody I, Antibody II, Antibody III, Antibody IV,
Antibody V, Antibody VI)
RASQSISSNLN
SEQ ID NO: 13
(LCDR2 of Antibody I, Antibody II, Antibody III, Antibody IV,
Antibody V, Antibody VI)
YAASSLQS
SEQ ID NO: 14
(LCDR3 of Antibody I, Antibody II, Antibody III, Antibody VI,
Antibody V, Antibody VI)
QQSYSIPWT
SEQ ID NO: 15
(HC of Antibody II)
X₄VQLLESGGGLVQPGGSLRLSCAASGFTFSSYSLSWVRQAPGKGLEWVSAISVS
GGSTYYADSVKGRFTISRDNSKNTLYLQMNSLRAEDTAVYYCARDDLIPTRGTF
YNWFDPWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEAAGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG
Wherein X₄ is E or pE
SEQ ID NO:16
(HCDNA of Antibody II)
gaggtgcagctgttggagtctgggggaggcttggtacagcctggggggtccctgagactctcctgtgcagcctctggattcacct
ttagcagctattccctgagctgggtccgccaggctccagggaaggggctggagtgggtctcagctattagtgtcagtggtggtag
cacatactacgcagactccgtgaagggtcggttcaccatctccagagacaattccaagaacacgctgtatctgcaaatgaacagc
ctgagagccgaggacacggccgtatattactgtgcgagagatgacctaataccaacgcgtggtaccttctacaactggttcgacc
cctggggccagggaaccctggtcaccgtctcctcagccagcaccaagggcccatcggtcttcccactagcgccctgctccagg
agcacctccgagagcacagccgccctgggctgcctggtcaaggactacttccccgagccggtgacggtgtcgtggaactcag
gagccctgaccagcggcgtgcacaccttcccggctgtcctacagtcctcaggactctactccctcagcagcgtggtgaccgtgc
cctccagcagcttgggcacgaagacctacacctgcaacgtagatcacaagcccagcaacaccaaggtggacaagagagttga
gtccaaatatggtcccccatgcccaccctgcccagcacctgaggccgccgggggaccatcagtcttcctgttccccccaaaacc
caaggacactctcatgatctcccggacccctgaggtaacgtgcgtggtggtggacgtgagccaggaagaccccgaggtccagt
tcaactggtacgtggatggcgtggaggtgcataatgccaagacaaagccgcgggaggagcagttcaacagcacgtaccgtgt
ggtcagcgtcctcaccgtcctgcaccaggactggctgaacggcaaggagtacaagtgcaaggtctccaacaaaggcctcccgt
cctccatcgagaaaaccatctccaaagccaaagggcagccccgagagccacaggtgtacaccctgcccccatcccaggagga
gatgaccaagaaccaggtcagcctgacctgcctggtcaaaggcttctaccccagcgacatcgccgtggagtgggaaagcaatg
ggcagccggagaacaactacaagaccacgcctcccgtgctggactccgacggctccttcttcctctacagcaggctaaccgtg
gacaagagcaggtggcaggaggggaatgtcttctcatgctccgtgatgcatgaggctctgcacaaccactacacacagaagag
cctctccctgtctctgggt
SEQ ID NO: 17
(VH of Antibody II)
X₄VQLLESGGGLVQPGGSLRLSCAASGFTFSSYSLSWVRQAPGKGLEWVSAISVS
GGSTYYADSVKGRFTISRDNSKNTLYLQMNSLRAEDTAVYYCARDDLIPTRGTF
YNWFDPWGQGTLVTVSS
Wherein X₄ is E or pE
SEQ ID NO: 18
(HC of Antibody III)
X₄VQLLESGGGLVQPGGSLRLSCAASGFTFSSYSLSWVRQAPGKGLEWVSAISVS
GGSTYYADSVKGRFTISRDNSKNTLYLQMNSLRAEDTAVYYCARDDLITTRGTF
YNWFDPWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEAAGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG
Wherein X₄ is E or pE
SEQ ID NO: 19
(HCDNA of Antibody III)
gaggtgcagctgttggagtctgggggaggcttggtacagcctggggggtccctgagactctcctgtgcagcctctggattcacct
ttagcagctattccttgagctgggtccgccaggctccagggaaggggctggagtgggtctcagctattagtgtcagtggtggtag
cacatactacgcagactccgtgaagggtcggttcaccatctccagagacaattccaagaacacgctgtatctgcaaatgaacagc
ctgagagccgaggacacggccgtatattactgtgcgagagatgacctaataacaacgcgtggtaccttctacaactggttcgacc
cctggggccagggaaccctggtcaccgtctcctcagcctccaccaagggcccatcggtcttcccgctagcgccctgctccagg
agcacctccgagagcacagccgccctgggctgcctggtcaaggactacttccccgaaccggtgacggtgtcgtggaactcag
gcgccctgaccagcggcgtgcacaccttcccggctgtcctacagtcctcaggactctactccctcagcagcgtggtgaccgtgc
cctccagcagcttgggcacgaagacctacacctgcaacgtagatcacaagcccagcaacaccaaggtggacaagagagttga
gtccaaatatggtcccccatgcccaccctgcccagcacctgaggccgccgggggaccatcagtcttcctgttccccccaaaacc
caaggacactctcatgatctcccggacccctgaggtcacgtgcgtggtggtggacgtgagccaggaagaccccgaggtccagt
tcaactggtacgtggatggcgtggaggtgcataatgccaagacaaagccgcgggaggagcagttcaacagcacgtaccgtgt
ggtcagcgtcctcaccgtcctgcaccaggactggctgaacggcaaggagtacaagtgcaaggtctccaacaaaggcctcccgt
cctccatcgagaaaaccatctccaaagccaaagggcagccccgagagccacaggtgtacaccctgcccccatcccaggagga
gatgaccaagaaccaggtcagcctgacctgcctggtcaaaggcttctaccccagcgacatcgccgtggagtgggaaagcaatg
ggcagccggagaacaactacaagaccacgcctcccgtgctggactccgacggctccttcttcctctacagcaggctaaccgtg
gacaagagcaggtggcaggaggggaatgtcttctcatgctccgtgatgcatgaggctctgcacaaccactacacacagaagag
cctctccctgtctctgggt
SEQ ID NO: 20
(HC of Antibody V)
X₄VQLLESGGGLVQPGGSLRLSCAASGFTFSSYSLSWVRQAPGKGLEWVSAISVS
GGSTYYADSVKGRFTISRDNSKNTLYLQMNSLRAEDTAVYYCARDDLITTRGTF
YNWFDPWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTK
VDKRVEPKSCDKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCS
VMHEALHNHYTQKSLSLSPGK
Wherein X₄ is E or pE
SEQ ID NO: 21
(HC DNA of Antibody V)
gaggtgcagctgttggagtctgggggaggcttggtacagcctggggggtccctgagactctcctgtgcagcctctggattcacct
ttagcagctattccttgagctgggtccgccaggctccagggaaggggctggagtgggtctcagctattagtgtcagtggtggtag
cacatactacgcagactccgtgaagggtcggttcaccatctccagagacaattccaagaacacgctgtatctgcaaatgaacagc
ctgagagccgaggacacggccgtatattactgtgcgagagatgacctaataacaacgcgtggtaccttctacaactggttcgacc
cctggggccagggaaccctggtcaccgtctcctcagctagcaccaagggcccatcggtcttccccctggcaccctcctccaaga
gcacctctgggggcacagcggccctgggctgcctggtcaaggactacttccccgaaccagtgacggtgtcgtggaactcaggc
gcactgaccagcggcgtgcacaccttcccggctgtcctacagtcctcaggactctactccctcagcagcgtggtgaccgtgccct
ccagcagcttgggcacccagacctacatctgcaacgtgaatcacaagcccagcaacaccaaggtggacaagagagttgagcc
caaatcttgtgacaaaactcacacatgcccaccgtgcccagcacctgaagccgccgggggaccgtcagtcttcctcttcccccc
aaaacccaaggacaccctcatgatctcccggacccctgaggtcacatgcgtggtggtggacgtgagccacgaagaccctgag
gtcaagttcaactggtatgtggacggcgtggaggtgcataatgccaagacaaagccgcgggaggagcagtacaacagcacgt
accgtgtggtcagcgtcctcaccgtcctgcaccaagactggctgaatggcaaggagtacaagtgcaaggtctccaacaaagcc
ctcccagcccccatcgagaaaaccatctccaaagccaaagggcagccccgagaaccacaggtgtacaccctgcccccatccc
gggaggagatgaccaagaaccaagtcagcctgacctgcctggtcaaaggcttctatcccagcgacatcgccgtggagtggga
gagcaatgggcagccggagaacaactacaagaccacgcctcccgtgctggactccgacggctccttcttcctctattccaagct
caccgtggacaagagcaggtggcagcaggggaacgtcttctcatgctccgtgatgcatgaggctctgcacaaccactacacgc
agaagagcctctccctgtctccgggcaaa
SEQ ID NO: 22
(VH of Antibody III, Antibody V)
X₄VQLLESGGGLVQPGGSLRLSCAASGFTFSSYSLSWVRQAPGKGLEWVSAISVS
GGSTYYADSVKGRFTISRDNSKNTLYLQMNSLRAEDTAVYYCARDDLITTRGTF
YNWFDPWGQGTLVTVSS
Wherein X₄ is E or pE
SEQ ID NO: 23
HC Antibody VI
X₄VQLLESGGGLVQPGGSLRLSCAASGFTFSSYSFSWVRQAPGKGLEWVSAISVS
GGSTYYADSVKGRFTISRDNSKNTLYLQMNSLRAEDTAVYYCARDDLITTRGTF
YNWFDPWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTK
VDKRVEPKSCDKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCS
VMHEALHNHYTQKSLSLSPGK
Wherein X₄ is E or pE
SEQ ID NO: 24
HC DNA Antibody VI
gaggtgcagctgttggagtctgggggaggcttggtacagcctggggggtccctgagactctcctgtgcagcctctggattcacct
ttagcagctattccttcagctgggtccgccaggctccagggaaggggctggagtgggtctcagctattagtgtcagtggtggtag
cacatactacgcagactccgtgaagggtcggttcaccatctccagagacaattccaagaacacgctgtatctgcaaatgaacagc
ctgagagccgaggacacggccgtatattactgtgcgagagatgacctaataacaacgcgtggtaccttctacaactggttcgacc
cctggggccagggaaccctggtcaccgtctcctcagctagcaccaagggcccatcggtcttccccctggcaccctcctccaaga
gcacctctgggggcacagcggccctgggctgcctggtcaaggactacttccccgaaccggtgacggtgtcgtggaactcagg
cgccctgaccagcggcgtgcacaccttcccggctgtcctacagtcctcaggactctactccctcagcagcgtggtgaccgtgcc
ctccagcagcttgggcacccagacctacatctgcaacgtgaatcacaagcccagcaacaccaaggtggacaagagagttgag
cccaaatcttgtgacaaaactcacacatgcccaccgtgcccagcacctgaagccgccgggggaccgtcagtcttcctcttcccc
ccaaaacccaaggacaccctcatgatctcccggacccctgaggtcacatgcgtggtggtggacgtgagccacgaagaccctga
ggtcaagttcaactggtatgtggacggcgtggaggtgcataatgccaagacaaagccgcgggaggagcagtacaacagcacg
taccgtgtggtcagcgtcctcaccgtcctgcaccaagactggctgaatggcaaggagtacaagtgcaaggtctccaacaaagcc
ctcccagcccccatcgagaaaaccatctccaaagccaaagggcagccccgagaaccacaggtgtacaccctgcccccatccc
gggaggagatgaccaagaaccaagtcagcctgacctgcctggtcaaaggcttctatcccagcgacatcgccgtggagtggga
gagcaatgggcagccggagaacaactacaagaccacgcctcccgtgctggactccgacggctccttcttcctctattccaagct
caccgtggacaagagcaggtggcagcaggggaacgtcttctcatgctccgtgatgcatgaggctctgcacaaccactacacgc
agaagagcctctccctgtctccgggcaaa
SEQ ID NO: 25
(VH of Antibody VI)
X₄VQLLESGGGLVQPGGSLRLSCAASGFTFSSYSFSWVRQAPGKGLEWVSAISVS
GGSTYYADSVKGRFTISRDNSKNTLYLQMNSLRAEDTAVYYCARDDLITTRGTF
YNWFDPWGQGTLVTVSS
Wherein X₄ is E or pE
SEQ ID NO: 26
(Human IL-1B)
MAEVPELASEMMAYYSGNEDDLFFEADGPKQMKCSFQDLDLCPLDGGIQLRISD
HHYSKGFRQAASVVVAMDKLRKMLVPCPQTFQENDLSTFFPFIFEEEPIFFDTWD
NEAYVHDAPVRSLNCTLRDSQQKSLVMSGPYELKALHLQGQDMEQQVVFSMSF
VQGEESNDKIPVALGLKEKNLYLSCVLKDDKPTLQLESVDPKNYPKKKMEKRFV
FNKIEINNKLEFESAQFPNWYISTSQAENMPVFLGGTKGGQDITDFTMQFVSS
SEQ ID NO: 27
(mature human IL-1B protein; residues 117-269 of SEQ ID NO: 19)
APVRSLNCTLRDSQQKSLVMSGPYELKALHLQGQDMEQQVVFSMSFVQGEESN
DKIPVALGLKEKNLYLSCVLKDDKPTLQLESVDPKNYPKKKMEKRFVFNKIEINN
KLEFESAQFPNWYISTSQAENMPVFLGGTKGGQDITDFTMQFVSS
SEQ ID NO: 28
(canakinumab HC)
QVQLVESGGGVVQPGRSLRLSCAASGFTFSVYGMNWVRQAPGKGLEWVAIIWY
DGDNQYYADSVKGRFTISRDNSKNTLYLQMNGLRAEDTAVYYCARDLRTGPFD
YWGQGTLVTVSSASTKGPSVFPLAPCSRSTSESTAALGCLVKDYFPEPVTVSWNS
GALTSGVHTFPAVLQSSGLYSLSSVVTVPSSSLGTKTYTCNVDHKPSNTKVDKRV
ESKYGPPCPPCPAPEAAGGPSVFLFPPKPKDTLMISRTPEVTCVVVDVSQEDPEVQ
FNWYVDGVEVHNAKTKPREEQFNSTYRVVSVLTVLHQDWLNGKEYKCKVSNK
GLPSSIEKTISKAKGQPREPQVYTLPPSQEEMTKNQVSLTCLVKGFYPSDIAVEWE
SNGQPENNYKTTPPVLDSDGSFFLYSRLTVDKSRWQEGNVFSCSVMHEALHNHY
TQKSLSLSLG
SEQ ID NO: 29
(canakinumab LC)
EIVLTQSPDFQSVTPKEKVTITCRASQSIGSSLHWYQQKPDQSPKLLIKYASQSFSG
VPSRFSGSGSGTDFTLTINSLEAEDAAAYYCHQSSSLPFTFGPGTKVDIKRTVAAP
SVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC

1. An antibody that binds human Protein IL-1β (SEQ ID NO:27), comprising a heavy chain variable region (VH) and a light chain variable region (VL), wherein the VH comprises heavy chain complementarity determining regions (HCDR) HCDR1, HCDR2, and HCDR3, and the VL comprises light chain complementarity determining regions (LCDR) LCDR1, LCDR2, and LCDR3, wherein
the HCDR1 comprises AASGFTFSSYSX₁S, wherein X₁ is Phe or Leu (SEQ ID NO:8),
the HCDR2 comprises AISVSGGSTYYAX₂SVKG, wherein X₂ is Pro or Asp (SEQ ID NO:9),
the HCDR3 comprises ARDDLIX₃TRGTFYNWFDP, wherein X₃ is Thr or Pro (SEQ ID NO:10),
the LCDR1 comprises RASQSISSNLN (SEQ ID NO:12),
the LCDR2 comprises YAASSLQS (SEQ ID NO:13), and
the LCDR3 comprises QQSYSIPWT (SEQ ID NO:14).

2. The antibody of claim 1, wherein
the HCDR1 comprises AASGFTFSSYSX₁S (SEQ ID NO:8), wherein X₁ is Phe,
the HCDR2 comprises AISVSGGSTYYAX₂SVKG (SEQ ID NO:9), wherein X₂ is Pro,
the HCDR3 comprises ARDDLIX₃TRGTFYNWFDP (SEQ ID NO:10), wherein X₃ is Thr.

3. The antibody of claim 1, wherein
the HCDR1 comprises AASGFTFSSYSX₁S (SEQ ID NO:8), wherein X₁ is Leu,
the HCDR2 comprises AISVSGGSTYYAX₂SVKG (SEQ ID NO:9), wherein X₂ is Asp,
the HCDR3 comprises ARDDLIX₃TRGTFYNWFDP (SEQ ID NO:10), wherein X₃ is Pro.

4. The antibody of claim 1, wherein
the HCDR1 comprises AASGFTFSSYSX₁S (SEQ ID NO:8), wherein X₁ is Leu,
the HCDR2 comprises AISVSGGSTYYAX₂SVKG (SEQ ID NO:9), wherein X₂ is Asp,
the HCDR3 comprises ARDDLIX₃TRGTFYNWFDP (SEQ ID NO:10), wherein X₃ is Thr.

5. The antibody of claim 1, wherein
the HCDR1 comprises AASGFTFSSYSX₁S (SEQ ID NO:8), wherein X₁ is Phe,
the HCDR2 comprises AISVSGGSTYYAX₂SVKG (SEQ ID NO:9), wherein X₂ is Asp,
the HCDR3 comprises ARDDLIX₃TRGTFYNWFDP (SEQ ID NO:10), wherein X₃ is Thr.

6. The antibody of claim 1 wherein the VH comprises SEQ ID NO:7 and the VL comprises SEQ ID NO:11.

7. The antibody of claim 1, wherein the antibody comprises a heavy chain (HC) comprising a sequence selected from SEQ ID NO:1 or SEQ ID NO:3 and a light chain (LC) comprising SEQ ID NO:5.

8. The antibody of claim 1 wherein the antibody comprises a HC comprising amino acids 2-451 selected from SEQ ID NO:1 or SEQ ID NO:3 and a LC comprising SEQ ID NO:5.

9. The antibody of claim 1 wherein the antibody comprises a HC consisting of a sequence selected from SEQ ID NO:1 or SEQ ID NO:3 and a LC consisting of SEQ ID NO:5.

10. The antibody of claim 1 wherein the VH comprises SEQ ID NO:17 and the VL comprises SEQ ID NO:11.

11. The antibody of claim 1 wherein the antibody comprises a HC comprising SEQ ID NO:15 and a LC comprising SEQ ID NO:5.

12. The antibody of claim 1 wherein the antibody comprises a HC comprising amino acids 2-451 of SEQ ID NO:15 and a LC comprising SEQ ID NO:5.

13. The antibody of claim 1 wherein the antibody comprises a HC consisting of SEQ ID NO:15 and a LC consisting of SEQ ID NO:5.

14. The antibody of claim 1 wherein the VH comprises SEQ ID NO:22 and the VL comprises SEQ ID NO:11.

15. The antibody of claim 1 wherein the antibody comprises a HC comprising a sequence selected from SEQ ID NO:18 or SEQ ID NO:20 and a LC comprising SEQ ID NO:5.

16. The antibody of claim 1 wherein the antibody comprises a HC comprising amino acids 2-451 selected from SEQ ID NO:18 or SEQ ID NO:20 and a LC comprising SEQ ID NO:5.

17. The antibody of claim 1 wherein the antibody comprises a HC consisting of a sequence selected from SEQ ID NO:18 or SEQ ID NO:20 and a LC consisting of SEQ ID NO:5.

18. The antibody of claim 1 wherein the VH comprises SEQ ID NO:25 and the VL comprises SEQ ID NO:11.

19. The antibody of claim 1 wherein the antibody comprises a HC comprising SEQ ID NO:23 and a LC comprising SEQ ID NO:5.

20. The antibody of claim 1 wherein the antibody comprises a HC comprising amino acids 2-451 of SEQ ID NO:23 and a LC comprising SEQ ID NO:5.

21. The antibody of claim 1 wherein the antibody comprises a HC consisting of a sequence of SEQ ID NO:23 and a LC consisting of SEQ ID NO:5.

22. The antibody of claim 1, wherein the antibody has a human IgG1 or IgG4 isotype.

23. The antibody of claim 22, wherein the antibody has a human IgG4 isotype.

24. The antibody of claim 22, wherein the antibody has a human IgG1 isotype.

25. A nucleic acid comprising a sequence encoding SEQ ID NO:1, SEQ ID NO:15, SEQ ID NO:18, SEQ ID NO:3, SEQ ID NO:20, or SEQ ID NO:23, and a second nucleic acid sequence encoding SEQ ID NO:5.

26. A composition comprising a first vector comprising a nucleic acid sequence encoding SEQ ID NO:1, SEQ ID NO:15, SEQ ID NO:18, SEQ ID NO:3, SEQ ID NO:20, or SEQ ID NO:23 and a second vector comprising a nucleic acid sequence encoding SEQ ID NO:5.

27. A cell comprising a first vector comprising a nucleic acid sequence encoding SEQ ID NO:1, SEQ ID NO:15, SEQ ID NO:18, SEQ ID NO:3, SEQ ID NO:20, or SEQ ID NO:23 and a second vector comprising a nucleic acid sequence encoding SEQ ID NO:5.

28. The cell of claim 27, wherein the cell is a mammalian cell.

29. A process of producing an antibody comprising culturing the cell of claim 27 under conditions such that the antibody is expressed and recovering the expressed antibody from the culture medium.

30. An antibody produced by culturing the cell by the process of claim 29.

31. A pharmaceutical composition comprising the antibody of, claim 1 and a pharmaceutically acceptable excipient, diluent or carrier.

32. A method of treating inflammatory disease in a subject in need thereof, comprising administering to the subject a therapeutically effective amount of the antibody of claim 1.

33. (canceled)

34. (canceled)

35. (canceled)

36. (canceled)

37. (canceled)


SEQUENCE LISTING (29 sequences)

SEQ ID NO: 1 - protein, 451 aa, synthetic construct
  REGION 1..451: note = Synthetic Construct
  VARIANT 1: note = MISC_FEATURE - X at position 1 is E or pyroglutamate
XVQLLESGGGLVQPGGSLRLSCAASGFTFSSYSFSWVRQAPGKGLEWVSAISVSGGSTYYAPSVKGRFTISRDNSKNTLYLQMNSLRAEDTAVYYCARDDLITTRGTFYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 2 - DNA, 1353 bp, synthetic construct
  misc_feature 1..1353: note = Synthetic Construct
  source 1..1353: mol_type = other DNA
gaggtgcagctgttggagtctgggggaggcttggtacagcctggggggtccctgagactctcctgtgcagcctctggattcacctttagcagctattccttcagctgggtccgccaggctccagggaaggggctggagtgggtctcagctattagtgtcagtggtggtagcacatactacgcaccctccgtgaagggtcggttcaccatctccagagacaattccaagaacacgctgtatctgcaaatgaacagcctgagagccgaggacacggccgtatattactgtgcgagagatgacctaataacaacgcgtggtaccttctacaactggttcgacccctggggccagggaaccctggtcaccgtctcctcagccagcaccaagggcccatcggtcttcccactagcgccctgctccaggagcacctccgagagcacagccgccctgggctgcctggtcaaggactacttccccgagccggtgacggtgtcgtggaactcaggagccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgaggccgccgggggaccatcagtcttcctgttccccccaaaacccaaggacactctcatgatctcccggacccctgaggtaacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggaaagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 3 - protein, 455 aa, synthetic construct
  REGION 1..455: note = Synthetic Construct
  VARIANT 1: note = MISC_FEATURE - X at position 1 is E or pyroglutamate
XVQLLESGGGLVQPGGSLRLSCAASGFTFSSYSFSWVRQAPGKGLEWVSAISVSGGSTYYAPSVKGRFTISRDNSKNTLYLQMNSLRAEDTAVYYCARDDLITTRGTFYNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 1365 bp, synthetic construct
  misc_feature 1..1365: note = Synthetic Construct
  source 1..1365: mol_type = other DNA
gaggtgcagctgttggagtctgggggaggcttggtacagcctggggggtccctgagactctcctgtgcagcctctggattcacctttagcagctattccttcagctgggtccgccaggctccagggaaggggctggagtgggtctcagctattagtgtcagtggtggtagcacatactacgcaccctccgtgaagggtcggttcaccatctccagagacaattccaagaacacgctgtatctgcaaatgaacagcctgagagccgaggacacggccgtatattactgtgcgagagatgacctaataacaacgcgtggtaccttctacaactggttcgacccctggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 5 - protein, 214 aa, synthetic construct
  REGION 1..214: note = Synthetic Construct
DIQMTQSPSSLSASVGDRVTITCRASQSISSNLNWYQQKPGKAPKLLIYAASSLQSGVPSRFSGSGSGTDFTLTISSLQPEDFATYYCQQSYSIPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - DNA, 642 bp, synthetic construct
  misc_feature 1..642: note = Synthetic Construct
  source 1..642: mol_type = other DNA
gacatccagatgacccagtctccatcctccctgtctgcatctgtaggagacagagtcaccatcacttgccgggcaagtcagagcattagcagcaatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtatcccttggacgttcggccaagggaccaaggtggaaatcaaacgaaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 7 - protein, 125 aa, synthetic construct
  REGION 1..125: note = Synthetic Construct
  VARIANT 1: note = MISC_FEATURE - Xaa at position 1 is E or pyroglutamate
XVQLLESGGGLVQPGGSLRLSCAASGFTFSSYSFSWVRQAPGKGLEWVSAISVSGGSTYYAPSVKGRFTISRDNSKNTLYLQMNSLRAEDTAVYYCARDDLITTRGTFYNWFDPWGQGTLVTVSS

SEQ ID NO: 8 - protein, 13 aa, synthetic construct
  REGION 1..13: note = Synthetic Construct
  VARIANT 12: note = MISC_FEATURE - X at position 12 is F or L
AASGFTFSSYSXS

SEQ ID NO: 9 - protein, 17 aa, synthetic construct
  REGION 1..17: note = Synthetic Construct
  VARIANT 13: note = MISC_FEATURE - X at position 13 is P or D
AISVSGGSTYYAXSVKG

SEQ ID NO: 10 - protein, 18 aa, synthetic construct
  REGION 1..18: note = Synthetic Construct
  VARIANT 7: note = MISC_FEATURE - X at position 7 is T or P
ARDDLIXTRGTFYNWFDP

SEQ ID NO: 11 - protein, 107 aa, synthetic construct
  REGION 1..107: note = Synthetic Construct
DIQMTQSPSSLSASVGDRVTITCRASQSISSNLNWYQQKPGKAPKLLIYAASSLQSGVPSRFSGSGSGTDFTLTISSLQPEDFATYYCQQSYSIPWTFGQGTKVEIK

SEQ ID NO: 12 - protein, 11 aa, synthetic construct
  REGION 1..11: note = Synthetic Construct
RASQSISSNLN

SEQ ID NO: 13 - protein, 8 aa, synthetic construct
  REGION 1..8: note = Synthetic Construct
YAASSLQS

SEQ ID NO: 14 - protein, 9 aa, synthetic construct
  REGION 1..9: note = Synthetic Construct
QQSYSIPWT

SEQ ID NO: 15 - protein, 451 aa, synthetic construct
  REGION 1..451: note = Synthetic Construct
  VARIANT 1: note = MISC_FEATURE - X at position 1 is E or pyroglutamate
XVQLLESGGGLVQPGGSLRLSCAASGFTFSSYSLSWVRQAPGKGLEWVSAISVSGGSTYYADSVKGRFTISRDNSKNTLYLQMNSLRAEDTAVYYCARDDLIPTRGTFYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 16 - DNA, 1353 bp, synthetic construct
  misc_feature 1..1353: note = Synthetic Construct
  source 1..1353: mol_type = other DNA
gaggtgcagctgttggagtctgggggaggcttggtacagcctggggggtccctgagactctcctgtgcagcctctggattcacctttagcagctattccctgagctgggtccgccaggctccagggaaggggctggagtgggtctcagctattagtgtcagtggtggtagcacatactacgcagactccgtgaagggtcggttcaccatctccagagacaattccaagaacacgctgtatctgcaaatgaacagcctgagagccgaggacacggccgtatattactgtgcgagagatgacctaataccaacgcgtggtaccttctacaactggttcgacccctggggccagggaaccctggtcaccgtctcctcagccagcaccaagggcccatcggtcttcccactagcgccctgctccaggagcacctccgagagcacagccgccctgggctgcctggtcaaggactacttccccgagccggtgacggtgtcgtggaactcaggagccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgaggccgccgggggaccatcagtcttcctgttccccccaaaacccaaggacactctcatgatctcccggacccctgaggtaacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggaaagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 17 - protein, 125 aa, synthetic construct
  REGION 1..125: note = Synthetic Construct
  VARIANT 1: note = MISC_FEATURE - X at position 1 is E or pyroglutamate
XVQLLESGGGLVQPGGSLRLSCAASGFTFSSYSLSWVRQAPGKGLEWVSAISVSGGSTYYADSVKGRFTISRDNSKNTLYLQMNSLRAEDTAVYYCARDDLIPTRGTFYNWFDPWGQGTLVTVSS

SEQ ID NO: 18 - protein, 451 aa, synthetic construct
  REGION 1..451: note = Synthetic Construct
  VARIANT 1: note = MISC_FEATURE - X at position 1 is E or pyroglutamate
XVQLLESGGGLVQPGGSLRLSCAASGFTFSSYSLSWVRQAPGKGLEWVSAISVSGGSTYYADSVKGRFTISRDNSKNTLYLQMNSLRAEDTAVYYCARDDLITTRGTFYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 19 - DNA, 1353 bp, synthetic construct
  misc_feature 1..1353: note = Synthetic Construct
  source 1..1353: mol_type = other DNA
gaggtgcagctgttggagtctgggggaggcttggtacagcctggggggtccctgagactctcctgtgcagcctctggattcacctttagcagctattccttgagctgggtccgccaggctccagggaaggggctggagtgggtctcagctattagtgtcagtggtggtagcacatactacgcagactccgtgaagggtcggttcaccatctccagagacaattccaagaacacgctgtatctgcaaatgaacagcctgagagccgaggacacggccgtatattactgtgcgagagatgacctaataacaacgcgtggtaccttctacaactggttcgacccctggggccagggaaccctggtcaccgtctcctcagcctccaccaagggcccatcggtcttcccgctagcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggaaagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 20 - protein, 455 aa, synthetic construct
  REGION 1..455: note = Synthetic Construct
  VARIANT 1: note = MISC_FEATURE - X at position 1 is E or pyroglutamate
XVQLLESGGGLVQPGGSLRLSCAASGFTFSSYSLSWVRQAPGKGLEWVSAISVSGGSTYYADSVKGRFTISRDNSKNTLYLQMNSLRAEDTAVYYCARDDLITTRGTFYNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 1365 bp, synthetic construct
  misc_feature 1..1365: note = Synthetic Construct
  source 1..1365: mol_type = other DNA
gaggtgcagctgttggagtctgggggaggcttggtacagcctggggggtccctgagactctcctgtgcagcctctggattcacctttagcagctattccttgagctgggtccgccaggctccagggaaggggctggagtgggtctcagctattagtgtcagtggtggtagcacatactacgcagactccgtgaagggtcggttcaccatctccagagacaattccaagaacacgctgtatctgcaaatgaacagcctgagagccgaggacacggccgtatattactgtgcgagagatgacctaataacaacgcgtggtaccttctacaactggttcgacccctggggccagggaaccctggtcaccgtctcctcagctagcaccaagggcccatcggtcttccccctggcaccctcctccaagagcacctctgggggcacagcggccctgggctgcctggtcaaggactacttccccgaaccagtgacggtgtcgtggaactcaggcgcactgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 22 - protein, 125 aa, synthetic construct
  REGION 1..125: note = Synthetic Construct
  VARIANT 1: note = MISC_FEATURE - X at position 1 is E or pyroglutamate
XVQLLESGGGLVQPGGSLRLSCAASGFTFSSYSLSWVRQAPGKGLEWVSAISVSGGSTYYADSVKGRFTISRDNSKNTLYLQMNSLRAEDTAVYYCARDDLITTRGTFYNWFDPWGQGTLVTVSS

SEQ ID NO: 23 - protein, 455 aa, synthetic construct
  REGION 1..455: note = Synthetic Construct
  VARIANT 1: note = MISC_FEATURE - X at position 1 is E or pyroglutamate
XVQLLESGGGLVQPGGSLRLSCAASGFTFSSYSFSWVRQAPGKGLEWVSAISVSGGSTYYADSVKGRFTISRDNSKNTLYLQMNSLRAEDTAVYYCARDDLITTRGTFYNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - DNA, 1365 bp, synthetic construct
  misc_feature 1..1365: note = Synthetic Construct
  source 1..1365: mol_type = other DNA
gaggtgcagctgttggagtctgggggaggcttggtacagcctggggggtccctgagactctcctgtgcagcctctggattcacctttagcagctattccttcagctgggtccgccaggctccagggaaggggctggagtgggtctcagctattagtgtcagtggtggtagcacatactacgcagactccgtgaagggtcggttcaccatctccagagacaattccaagaacacgctgtatctgcaaatgaacagcctgagagccgaggacacggccgtatattactgtgcgagagatgacctaataacaacgcgtggtaccttctacaactggttcgacccctggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 25 - protein, 125 aa, synthetic construct
  REGION 1..125: note = Synthetic Construct
  VARIANT 1: note = MISC_FEATURE - X at position 1 is E or pyroglutamate
XVQLLESGGGLVQPGGSLRLSCAASGFTFSSYSFSWVRQAPGKGLEWVSAISVSGGSTYYADSVKGRFTISRDNSKNTLYLQMNSLRAEDTAVYYCARDDLITTRGTFYNWFDPWGQGTLVTVSS

SEQ ID NO: 26 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 27 - protein, 153 aa, Homo sapiens
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 28 - protein, 444 aa, synthetic construct
  REGION 1..444: note = Synthetic Construct
QVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - protein, 214 aa, synthetic construct
  REGION 1..214: note = Synthetic Construct
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC